(19) United States
(12) Patent Application Publication
(10) Pub. No.: US 2026/0345284 A1
(43) Pub. Date: Nov. 5, 2026

(54) Systems and Methods for Evacuating Surgical Smoke and Liquid from a Treatment Site

(71) Applicant: Stryker European Operations Limited, Cork (IE)

(72) Inventors: David Eustace, Cork (IE); Bruce McKee, Cork (IE)

(21) Appl. No.: 19/488,105

(22) PCT Filed: Dec. 31, 2024
(86) PCT No.: PCT/IB2024/000760

§ 371 Date: Nov. 26, 2025

Related U.S. Application Data
(60) Provisional application No. 63/621,446, filed on Jan. 16, 2024.

(51) Int. Cl. A61B 90/00

(52) U.S. Cl. CPC A61B 90/08; A61B 2218/008

(57) ABSTRACT

In an example, a smoke and liquid separator device includes a separator housing that defines an internal chamber. The separator housing includes: (i) an inlet configured to receive into the internal chamber a liquid and a gas from the suction device, (ii) a gas outlet configured to output the gas from the internal chamber to a smoke mitigation system, and (iii) a liquid outlet configured to output the liquid from the internal chamber to a liquid disposal system. The smoke and liquid separator device also includes a valve assembly that is actuatable between (i) a closed state in which the valve assembly inhibits the liquid from passing through the liquid outlet, and (ii) an open state in which the valve assembly allows the liquid to pass through the liquid outlet.

### CROSS-REFERENCE TO RELATED APPLICATIONS

The present application claims the benefit of priority of U.S. Provisional Application No. 63/621,446, filed Jan. 16, 2024, the contents of which are hereby incorporated by reference in their entirety.

### FIELD

The present disclosure relates to fluid evacuation devices and methods and, in particular, to systems and method for evacuating surgical smoke and liquid from a treatment site (e.g., a surgical site).

### BACKGROUND

Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room.

Additionally, it may be desirable to evacuate liquids from the surgical site during some surgical procedures (e.g., to control bleeding and maintain a light of sight). For this reason, a liquid evacuation system may also be used during the surgical procedure. In general, the liquid evacuation system may also include a suction pump that can generate sufficient suction and/or vacuum pressure to draw the liquids away from the surgical site.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative implementation of the present disclosure when read in conjunction with the accompanying figures, wherein:

FIG. 1 depicts a simplified block diagram of a system for evacuating surgical smoke and a liquid, according to an example.

FIG. 2A shows a perspective view of a smoke-liquid separator, according to an example.

FIG. 2B shows a cross-sectional view of the smoke-liquid separator shown in FIG. 2A with a valve assembly in a closed state, according to an example.

FIG. 2C shows an enlarged view of a portion of the valve assembly shown in FIG. 2B, according to an example.

FIG. 2D shows a cross-sectional view of the smoke-liquid separator shown in FIG. 2B with the valve assembly in an open state, according to an example.

FIG. 2E shows an enlarged view of a portion of the valve assembly shown in FIG. 2D.

FIG. 3A shows a perspective view of a smoke-liquid separator, according to another example.

FIG. 3B shows the smoke-liquid separator of FIG. 3A with a valve assembly in a closed state, according to an example.

FIG. 3C shows the smoke-liquid separator of FIG. 3A with the valve assembly 140 in an open state, according to an example.

FIG. 4A shows a perspective view of the smoke-liquid separator, according to another example.

FIG. 4B shows a cross-sectional view of the smoke-liquid separator of FIG. 4A with a valve assembly in a closed state, according to an example.

FIG. 4C shows a cross-sectional view of the smoke-liquid separator of FIG. 4A with the valve assembly in an open state, according to the example.

FIG. 5A shows a first implementation of the suction device with a suction sleeve in an extended position, according to an example.

FIG. 5B shows the suction device of FIG. 5A with the suction sleeve in a retracted position, according to an example.

FIG. 5C shows a second implementation of the suction device with an electrosurgical electrode in an extended position, according to an example.

FIG. 5D shows the suction device of FIG. 5C with the electrosurgical electrode in a retracted position, according to the example.

FIG. 6 shows a flowchart for a process of evacuating a surgical smoke and a liquid, according to an example.

### DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, it can be desirable to evacuate surgical smoke and liquids from a surgical site during a surgical procedure. Prior approaches to evacuating surgical smoke and liquids generally involve using two separate devices at the surgical site, including one device for evacuating the surgical smoke (e.g., an electrosurgical device with smoke evacuation features) and another device for evacuating the liquid (e.g., a Yankauer suction device). However, this creates a challenge as multiple devices are required at the surgical site to evacuating the surgical smoke and the liquids, requiring multiple practioners to hold the multiple devices. This can be particularly challenging when the surgical procedure is a minimal invasive surgical procedure, which may involve working in relatively narrow and/or deep channels as the two separate devices can obstruct lines of sight and/or limit maneuverability at the surgical site.

There are several problems associated with evacuating both surgical smoke and liquids from a single device. As one example, conventional suction sources for smoke evacuation are typically not strong enough to evacuate fluid effectively and in a timely manner. Additionally, for example, if a liquid were to pass through the air flow pathway of a typical smoke mitigation system, the liquid may damage or impair a filter of the smoke mitigation system. Further, for instance, conventional liquid suction systems typically do not include a filter or other means for trapping surgical smoke so the surgical smoke could migrate out of the liquid suction system, which would not mitigate the risks associated with exposure to surgical smoke.

The present application provides for systems and methods that can address one or more challenges associated with evacuating surgical smoke and liquids from a treatment site. In particular, the present disclosure provides for systems and methods that involve a smoke-liquid separator, which can (i) receive both surgical smoke and liquid evacuated by a suction device from the treatment site, (ii) separate, using the smoke-liquid separator, the surgical smoke and the liquid from each other, (iii) direct the surgical smoke to a smoke mitigation system, and (iv) direct the liquid to a liquid disposal system. Within examples, the smoke-liquid separator can receive the surgical smoke and the liquid in an internal chamber of a housing, and the smoke-liquid separator can include a valve assembly that can automatically control when the smoke mitigation system and/or the liquid disposal system are fluidly coupled to the internal chamber to assist in separating and outputting the surgical smoke and the liquid to the smoke mitigation system and the liquid disposal system, respectively. Additionally, within examples, the valve assembly can automatically perform such control functions based on an amount of the liquid in the internal chamber.

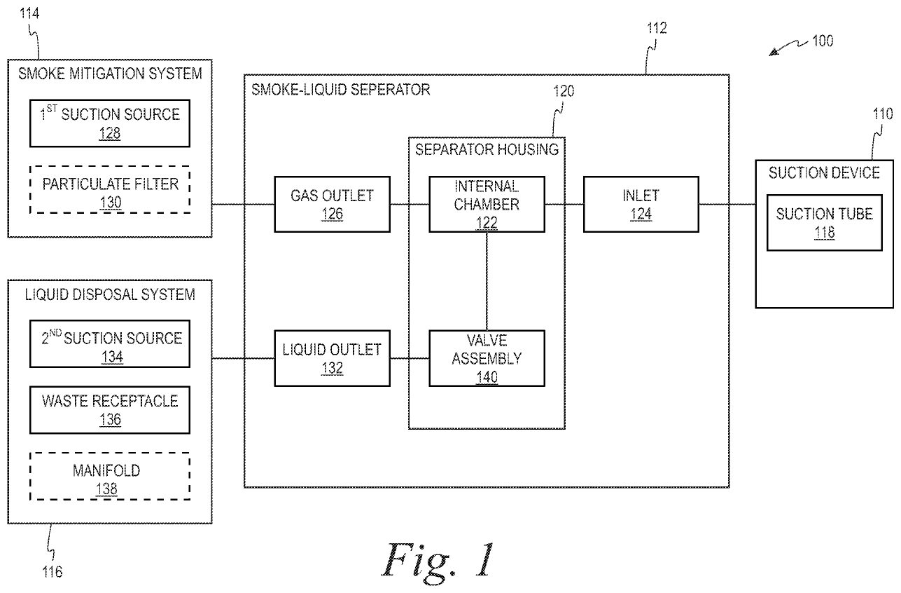

Referring to FIG. 1, a system 100 for evacuating a surgical smoke and a liquid from a treatment site (e.g., a surgical site) is shown according to an example. As shown in FIG. 1, the system 100 can include a suction device 110, a smoke-liquid separator 112, a smoke mitigation system 114, and a liquid disposal system 116.

The suction device 110 is a configured to evacuate a surgical smoke and a liquid from a surgical site. For example, the suction device 110 can include a suction tube 118. The suction tube 118 can have a proximal end and a distal end. As described in further detail below, the proximal end of the suction tube 118 can be coupled to the smoke-liquid separator and receive suction from the smoke-liquid separator 112. The distal end of the suction tube 118 can be positioned at the surgical site to use the suction to receive the surgical smoke and the liquid into a lumen of the suction tube. In this way, the suction tube 118 can use the suction to capture the surgical smoke and the liquid at the surgical site, and evacuate the surgical smoke and the liquid along the suction tube 118 to the smoke-liquid separator 112.

In some examples, the suction device 110 can be a medical device that is further configured to create the surgical smoke during a surgical procedure. For instance, the suction device 110 can include the suction tube 118 and an electrosurgical device that can apply a radiofrequency (RF) energy to tissue at the surgical site to cut and/or coagulate tissue. In some implementations, the electrosurgical device can be a monopolar electrosurgical pencil and/or a bipolar electrosurgical device that includes one or more electrosurgical electrodes that can apply the RF energy to the tissue, and the suction tube 118. In another example, the suction device 110 can include the suction tube 118 and a laser device that can apply a laser light to vaporize and/or ablate the tissue. In another example, the suction device 110 can include the suction tube 118 and an ultrasonic device that can use high-frequency vibrations to cut and/or dissect tissue. In another example, the suction device 110 can include the suction tube 118 and a drill that can cut bone. In another example, the suction tube 118 can include the suction tube 118 and a saw that can cut tissue or bone.

Accordingly, within examples, the suction device 110 can include features that can create the surgical smoke as a by-product of an interaction between the suction device 110 and the tissue at the surgical site, and features that can evacuate the surgical smoke and the liquids at the surgical site. This can help to address at least some of the challenges described above, which are associated with performing a surgical procedure using multiple, separate devices to work on the tissue, evacuate the surgical smoke, and evacuate the liquids at the surgical site.

In other examples, the suction device 110 can additionally or alternatively include one or more features that can perform one or more non-suction functions, which may not generate the surgical smoke. For instance, the suction device 110 can include an ultrasonic aspirator for removing tissue (e.g., using ultrasonic frequency vibrations generated by a piezoelectric element). Although the suction device 110 may not directly cause the surgical smoke to be generated during the surgical procedure, the multi-feature capabilities of the suction device 110 can also help to reduce a number of devices that are used by practioners at the surgical site.

While it can be beneficial for the suction device 110 to include one or more functions in addition to applying suction to evacuate the surgical smoke and the liquid from the surgical site, the suction device 110 can be a dedicated suction device that does not have any other function in still other examples.

In some examples, the suction device 110 can be a handheld device that can be grasped and manipulated by a practioner during the surgical procedure. In other examples, the suction device 110 can be an end effector of a robotic surgical system (e.g., the suction device 110 can be an end effector on a robotic arm that can be operated by a practioner using a remote control device and/or in an automated manner based on computer programming).

As shown in FIG. 1, the suction device 110 can be coupled to the smoke-liquid separator 112. The smoke-liquid separator 112 includes a separator housing 120 that defines an internal chamber 122. The separator housing 120 can be configured to contain in the internal chamber 122 the surgical smoke and the liquid received from the suction device 110. For instance, the separator housing 120 can be formed from a fluid impermeable material. The separator housing 120 can additionally or alternatively be formed from one or more materials that have sufficient rigidity to withstand suction pressures applied to the separator housing 120 by the smoke mitigation system 114 and/or the liquid disposal system 116. For instance, the separator housing 120 can be formed from a metal and/or a plastic. As examples, the separator housing 120 can be formed from one or more plastic materials selected from a group consisting of: polyethylene, polypropylene, and polycarbonate.

Within examples, the separator housing 120 can include one or more outer walls that form an enclosure, which defines the internal chamber 122. The one or more outer walls can include a top wall, a bottom wall, and one or more side walls. The top wall is located above the bottom wall when the smoke-liquid separator 112 is in use. In some examples, the bottom wall can be configured to support the separator housing 120 on a surface (e.g., a counter, a table, a floor, and/or a medical cart). In other examples, the separator housing 120 can additionally or alternatively be configured to couple to the smoke mitigation system 114 and/or the liquid disposal system 116 with the top wall located above the bottom wall. For instance, in one example implementation, the separator housing 120 can be coupled to a manifold of a waste management system that includes both the smoke mitigation system 114 and the liquid disposal system 116. One example of such a waste management system is the NEPTUNE 3 Waste Management System, which is currently sold by Stryker Corporation.

Within examples, an upper portion of the internal chamber 122 can be located above a lower portion of the internal chamber 122. For instance, upper portion of the internal chamber 122 can extend from the top wall of the separator housing 120 to an intermediate point on the one or more side walls, and the lower portion of the internal chamber 122 can extend from the bottom wall of the separator housing 120 to the intermediate point. As described in further examples, the intermediate point can be a point at which a level of the liquid in the internal chamber 122 causes the liquid to be evacuated out of the internal chamber 122.

The separator housing 120 includes an inlet 124 that is configured to fluidly couple the internal chamber 122 to the suction device 110, and receive into the internal chamber 122 the liquid and the surgical smoke from the suction device 110. For instance, the inlet 124 can include a first aperture in the one or more outer walls of the separator housing 120. In some examples, the inlet 124 can be coupled to the suction device 110 (e.g., the suction tube 118) by an input connector tube. To help couple the inlet 124 to the input connector tube, the inlet 124 can include at least one fitting selected from a group consisting of: a luer coupling, a push-on connector, a threaded connector, and a bayonet connector.

The separator housing 120 can also include a gas outlet 126 that is configured to fluidly couple the internal chamber 122 to the smoke mitigation system 114. The gas outlet 126 is configured to output the surgical smoke from the internal chamber 122 to the smoke mitigation system 114. For instance, the gas outlet 126 can include a second aperture in the one or more outer walls of the separator housing 120. In some examples, the gas outlet 126 be coupled to the smoke mitigation system 114 by a gas tube. To help couple the gas outlet 126 to the gas tube, the gas outlet 126 can include at least one fitting selected from a group consisting of: a luer coupling, a push-on connector, a threaded connector, and a bayonet connector.

The smoke mitigation system 114 can include a first suction source 128 that can apply a first suction force at the gas outlet 126 (e.g., via the gas tube). In some examples, the first suction source 128 can be configured to generate suction such that the first suction force at the gas outlet 126 is between approximately 0 millimeters of mercury (mm Hg) and approximately 90 mm Hg.

In some examples, the smoke mitigation system 114 can be coupled to an exhaust system (e.g., an in-wall exhaust system) that exhausts the surgical smoke out of an operating room. For instance, the smoke mitigation system 114 can include an exhaust vent that can exhaust the surgical smoke to an environment that is external to an environment in which the surgical procedure is being performed such that the practioners performing the surgical procedure are not exposed to the surgical smoke.

In other implementations, the smoke mitigation system 114 can include a particulate filter 130 that can receive the surgical smoke as an input, remove one or more particulates from the surgical smoke, and output filtered air (e.g., to the operating room). As examples, the particulate filter 130 can include one or more filters selected from a group consisting of: a high efficiency particulate air (HEPA) filter, an ultra-low particulate air (ULPA) filter, an efficiency particulate air (EPA) filter, and an activated carbon filter. In implementations in which the smoke mitigation system 114 includes the particulate filter 130, the first suction source 128 can generate the first suction force at a level that is suitable to evacuate the surgical smoke from the internal chamber 122 through the gas outlet 126, along the gas tube to the smoke mitigation system 114, and through the particulate filter 130.

The separator housing 120 can also include a liquid outlet 132 that is configured to fluidly couple the internal chamber 122 to the liquid disposal system 116. The liquid outlet 132 is configured to output the liquid from the internal chamber 122 to the liquid disposal system 116. For instance, the liquid outlet 132 can include a third aperture in the one or more outer walls of the separator housing 120.

In some examples, the liquid outlet 132 can be located on the separator housing 120 lower than the gas outlet 126. As such, the liquid outlet 132 can be closer to the bottom wall of the separator housing 120 than the gas outlet 126, and the gas outlet 126 can be closer to the top wall of the separator housing 120 than the liquid outlet 132. In some implementations, the liquid outlet 132 can be located at the lower portion of the internal chamber 122, and the gas outlet 126 can be located at the upper portion of the internal chamber 122. As described in further detail below, locating the liquid outlet 132 lower on the separator housing 120 than the gas outlet 126 can help to separate the surgical smoke and the liquid from each other.

In some examples, the liquid outlet 132 can be coupled to the liquid disposal system 116 by a flexible drain tube. In some implementations, the liquid outlet 132 can include at least one fitting selected from a group consisting of: a luer coupling, a push-on connector, a threaded connector, and a bayonet connector. In other examples, the liquid outlet 132 can additionally or alternatively be coupled to the liquid disposal system 116 by a substantially rigid structure such as, for instance, a drain housing. Examples that include a drain housing are described in further detail below.

The liquid disposal system 116 can include a second suction source 134 that can apply a second suction force at the liquid outlet 132 (e.g., via the drain tube and/or the drain housing). In some examples, the second suction source 134 can be configured to generate suction such that the second suction force at the liquid outlet 132 is between approximately 0 mm Hg and approximately 520 mm Hg.

In some implementations, the liquid disposal system 116 can include a waste receptacle 136 that can receive and contain the liquid. As examples, the waste receptacle 136 can be an closed container that can help to mitigate leakage and/or spill of the liquid during collection, transport, and/or disposal of the liquid. In some examples, the liquid disposal system 116 can further include a manifold 138 between the waste receptacle 136 and the liquid outlet 132. The manifold 138 can include a plurality of ports for coupling with medical devices that may be used during the surgical procedure. In implementations in which the liquid disposal system 116 includes the manifold 138, the liquid outlet 132 can be coupled to the manifold by the drain tube and/or the drain housing.

In general, the second suction source 134 can generate the second suction force at a level that is suitable to evacuate the liquid from the internal chamber 122 through the liquid outlet 132, along any intermediate structures (e.g., the drain tube, the drain housing, and/or the manifold 138) and to the waste receptacle 136. In some examples, the second suction force generated by the second suction source 134 can be greater than the first suction force generated by the first suction source 128. This may be helpful due to the liquid having a greater density, viscosity, and/or surface tension than the surgical smoke.

In some examples, the smoke mitigation system 114 and the liquid disposal system 116 can provided in separate housings, independently movable, and/or independently powered relative to each other. In other examples, the smoke mitigation system 114 and the liquid disposal system 116 can be integrated together in a common housing, powered by a common power source, and/or immovable relative to each other. One example of an integrated system that includes both the smoke mitigation system 114 and the liquid disposal system 116 is the NEPTUNE 3 Waste Management System, which is currently sold by Stryker Corporation.

As shown in FIG. 1, the smoke-liquid separator 112 also includes a valve assembly 140 that is actuatable between (i) a closed state in which the valve assembly 140 inhibits the liquid from passing through the liquid outlet 132, and (ii) an open state in which the valve assembly 140 allows the liquid to pass through the liquid outlet 132. Within examples, the valve assembly 140 can automatically actuate between the closed state and the open state based on an amount of the liquid in the internal chamber 122. For instance, the valve assembly 140 can: (i) automatically actuate to the closed state when an amount of the liquid in the internal chamber 122 is less than a threshold amount, and (ii) automatically actuate to the open state when the amount of the liquid in the internal chamber 122 is greater than the threshold amount.

In some examples, the valve assembly 140 can use a weight of the liquid in the internal chamber 122 as a trigger for actuating the valve assembly 140 between the closed state and the open state. For instance, the weight of the liquid can cause two or more components of the valve assembly 140 to move relative to each other to block and unblock the liquid outlet 132. In other examples, the valve assembly 140 can include a float that can rise and fall with a level of the liquid in the internal chamber 122. In this way, the movement of the float responsive to the level of the liquid in the internal chamber 122 can provide the trigger for actuating the valve assembly 140 between the closed state and the open state. For instance, the float can be coupled to a plug that can block the liquid outlet 132 when the valve assembly 140 is in the closed state, and unblock the liquid outlet 132 when the valve assembly 140 is in the open state.

As described above, the second suction force is applied to the internal chamber 122 through the liquid outlet 132. Accordingly, when the valve assembly 140 is in the closed state, the valve assembly 140 can also inhibit or prevent the second suction source 134 from applying the second suction force to the internal chamber 122 via the liquid outlet 132. When the valve assembly 140 is in the open state, the valve assembly 140 can allow the second suction source 134 to apply the second suction force to the internal chamber 122 via the liquid outlet 132.

In some examples, the first suction source 128 can apply the first suction force to the internal chamber 122 via the gas outlet 126 when the valve assembly 140 is in the open state and when the valve assembly 140 is in the closed state. This can allow for evacuation of the surgical smoke in both the open state and the closed state (e.g., at all times). In some implementations, the liquid outlet 132 can be located below and the gas outlet 126 can be located above a level of the liquid at which the liquid actuates the valve assembly 140 to the open state. This can assist in evacuating the surgical smoke through the gas outlet 126 while the liquid is simultaneously evacuated through the liquid outlet 132.

In other examples, the first suction source 128 can apply the first suction force to the internal chamber 122 through the gas outlet 126 when the valve assembly 140 is in the closed state, and the valve assembly 140 can inhibit or prevent the first suction source 128 from applying the first suction force to the internal chamber 122 through the gas outlet 126 when the valve assembly 140 is in the closed state. In such examples, only one of the surgical smoke and the liquid can be evacuated at one time. For instance, the valve assembly 140 can include a component (e.g., a float and/or a plug) that can move between (i) a lower position at which the component blocks the liquid outlet 132 and does not block the gas outlet 126, and (ii) an upper position at which the component blocks the gas outlet 126 and does not block the liquid outlet 132. An implementation in which only one of the surgical smoke and the liquid can be evacuated at one time may be beneficial, for example, as a safety feature in instances in which the first suction source 128 or the second suction source 134 are relatively low pressure sources.

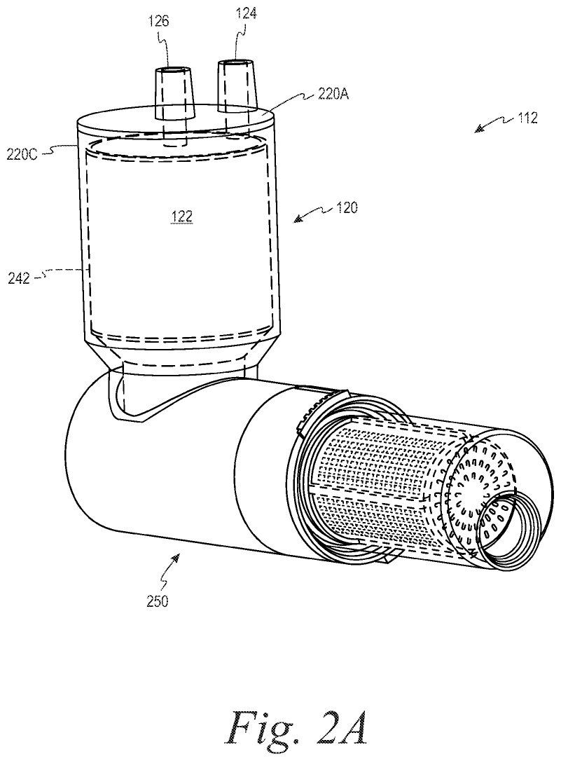

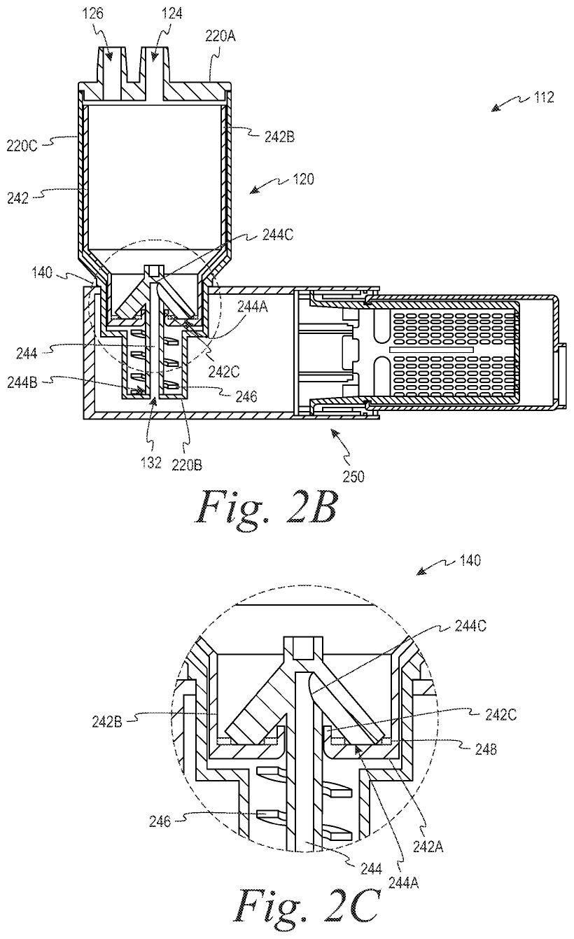

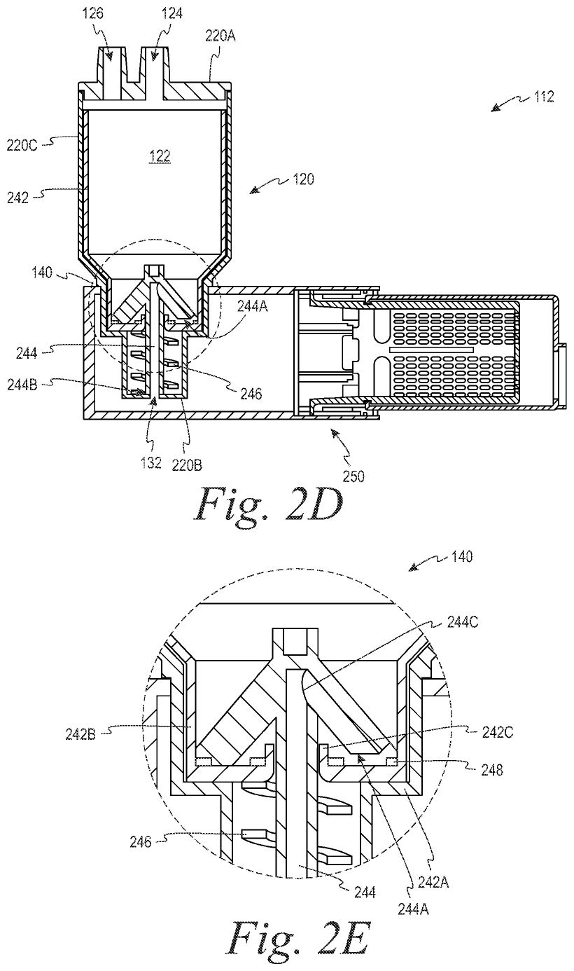

Referring now to FIGS. 2A-2E, an implementation of the smoke-liquid separator 112 is shown according to an example. In particular, FIG. 2A shows a perspective view of the smoke-liquid separator 112, FIG. 2B shows a cross-sectional view of the smoke-liquid separator 112 with the valve assembly 140 in the closed state, FIG. 2C shows an enlarged view of a portion of the valve assembly shown in FIG. 2B, FIG. 2D shows a cross-sectional view of the smoke-liquid separator 112 with the valve assembly 140 in the open state, and FIG. 2E shows an enlarged view of a portion of the valve assembly shown in FIG. 2D, according to the example.

As shown in FIGS. 2A-2E, the smoke-liquid separator 112 includes the separator housing 120 that defines the internal chamber 122. The separator housing 120 includes an inlet 124 that is configured to fluidly couple the internal chamber 122 to the suction device 110. The inlet 124 is configured to receive into the internal chamber 122 the liquid and the gas from the suction device 110. The separator housing 120 also includes the gas outlet 126 that is configured to output the gas from the internal chamber 122 to the smoke mitigation system 114, and the liquid outlet 132 that is configured to output the liquid from the internal chamber 122 to the liquid disposal system 116.

As shown in FIGS. 2A-2E, the separator housing 120 includes a top wall 220A, a bottom wall 220B, and a side wall 220C extending between the top wall 220A and the bottom wall 220B. In this example, the top wall 220A is a lid that is removably coupled to the side wall 220C (e.g., via a threaded coupling). However, in other examples, the top wall 220A can be non-removably coupled to the side wall 220C, or the top wall 220A and the side wall 220C can be a single, monolithic structure.

In this example, the inlet 124 and the gas outlet 126 are at the top wall 220A, and the liquid outlet 132 is at the bottom wall 220B. However, in other examples, the inlet 124 and/or the gas outlet 126 can be at respective apertures in the side wall 220C, which are located at the upper portion of the internal chamber 122 above the liquid outlet 132. Similarly, the liquid outlet 132 can be an aperture in the side wall 220C, which is located a lower portion of the internal chamber 122 below the inlet 124 and the gas outlet 126. With the inlet 124 located above the liquid outlet 132, the liquid moves from the inlet 124 toward the liquid outlet 132 due to a force of gravity.

As shown in FIGS. 2B-2E, the smoke-liquid separator 112 also includes the valve assembly 140 that is actuatable between (i) a closed state in which the valve assembly 140 inhibits the liquid from passing through the liquid outlet 132 (as shown in FIGS. 2B-2C), and (ii) an open state in which the valve assembly 140 allows the liquid to pass through the liquid outlet 132 (as shown in FIG. 2D-2E). As described above, the valve assembly 140 is configured to: (i) automatically actuate to the closed state when an amount of the liquid in the internal chamber 122 is less than a threshold amount, and (ii) automatically actuate to the open state when the amount of the liquid in the internal chamber 122 is greater than the threshold amount.

For example, in FIGS. 2B-2E, the valve assembly 140 includes a valve receptacle 242 that is disposed in the internal chamber 122 of the separator housing 120. The valve receptacle 242 is movable relative to the separator housing 120. For instance, the valve receptacle 242 can move up and down between a first position relative to the separator housing 120 shown in FIG. 2B and a second position relative to the separator housing 120 shown in FIG. 2D. As described in further detail below, in the first position of the valve receptacle 242 relative to the separator housing 120, the valve assembly 140 is in the closed state, and in the second position of the valve receptacle 242 relative to the separator housing 120, the valve assembly 140 is in the open state.

For example, the valve assembly 140 can also include a tube 244 that extends between a first tube end 244A and a second tube end 244B. The first tube end 244A can be in the internal chamber 122 of the separator housing 120 and the second tube end 244B can be in communication with the liquid outlet 132. For instance, in FIGS. 2B-2E, the second tube end 244B abuts against the bottom wall 220B of the separator housing 120 at the liquid outlet 132. In other examples, the tube 244 can extend through the liquid outlet 132 such that the second tube end 244B is external to the internal chamber 122.

As shown in FIG. 2B, when the valve assembly 140 is in the closed state, a wall 242A of the valve receptacle 242 contacts the first tube end 244A. In particular, the wall 242A of the valve receptacle 242 can block the first tube end 244A such that the liquid that accumulates in the lower portion of the internal chamber 122 (e.g., around the first tube end 244A) cannot enter the tube 244, and the second suction force applied to the liquid outlet 132 cannot propagate into the internal chamber 122.

As shown in FIG. 2D, when the valve assembly 140 is in the open state, the first tube end 244A is spaced apart from the wall 242A of the valve receptacle 242. For example, as shown in FIG. 2D, when greater than the threshold amount of the liquid is received in the valve receptacle 242, the weight of the liquid can cause the valve receptacle 242 to move downward toward the bottom wall 220B of the separator housing 120 (e.g., to the second position of the valve receptacle 242 relative to the separator housing 120). The tube 244 can be fixedly coupled to the separator housing 120 such that the valve receptacle 242 moves relative to the tube 244 when the valve receptacle 242 moves relative to the separator housing 120. As a result, when the valve assembly 140 is in the open state, a gap is formed between the first tube end 244A and the wall 242A of the valve receptacle 242. This gap allows the liquid to enter the tube 244 at the first tube end 244A, pass through the tube 244 to the second tube end 244B, and exit out of the internal chamber 122 via the liquid outlet 132 in communication with the second tube end 244B. Additionally, the gap between the first tube end 244A and the wall 242A allows the second suction source 134 to apply the second suction force to the internal chamber 122 through the liquid outlet 132 and the tube 244 to assist in evacuating the liquid from the internal chamber 122.

As shown in FIGS. 2B-2E, the valve assembly 140 can also include a spring 246 that biases the valve receptacle 242 towards the first position relative to the separator housing 120, and the first tube end 244A into contact with the wall 242A of the valve receptacle 242 (e.g., the spring 246 can bias the valve assembly 140 towards the closed state). For instance, in example shown in FIGS. 2B-2E, the spring 246 can include a first spring end that engages the bottom wall 220B of the separator housing 120, and a second spring end that the wall 242A of the valve receptacle 242. In this example, when the amount of the liquid in the internal chamber 122 is greater than the threshold amount, a weight of the liquid and the valve receptacle 242 overcomes a biasing force applied by the spring 246 between the separator housing 120 and the valve receptacle 242 to move the valve receptacle 242 to the second position in which a gap is formed between the first tube end 244A and the wall 242A. When at least a portion of the liquid has been evacuated through the liquid outlet 132 such that a weight of a remaining portion of the liquid in the internal chamber 122 is less than the threshold amount, the spring 246 can automatically return the valve receptacle 242 to the first position in which the first tube end 244A is blocked by the wall 242A and the valve assembly 140 is in the closed state.

As shown in FIGS. 2B-2E, the tube 244 can include a bend 244C between the first tube end 244A and the second tube end 244B. The bend 244C can help to orient the first tube end 244A and the second tube end 244B in a downward direction. In this example, the wall 242A of the valve receptacle 242 that engages and blocks the first tube end 244A can be a bottom wall of the valve receptacle 242. As shown in FIGS. 2A-2E, the valve receptacle 242 can further include an outer side wall 242B, an inner side wall 242C, and an opening at a top of the valve receptacle 242. The inner side wall 242C can extend upward from the wall 242A between respective portions of the tube 244 on opposing sides of the bend 244C. In this way, the valve receptacle 242 can be configured to receive and contain the liquid in the valve receptacle 242 until greater than the threshold amount of the liquid is present in the valve receptacle 242 to trigger the evacuation of the liquid through the tube 244 and the liquid outlet 132.

In some examples, the valve assembly 140 can also include one or more O-ring seals 248 at an interface between the first tube end 244A and the wall 242A of the valve receptacle 242 when the valve receptacle 242 is in the first position relative to the separator housing 120. This can help to inhibit the liquid entering the first tube end 244A of the tube 244 and/or the second suction source 134 applying the second suction pressure to the internal chamber 122 when the valve assembly 140 is in the closed state.

In FIGS. 2A-2E, the separator housing 120 is coupled to a drain housing 250, which is configured to couple to a manifold of the liquid disposal system 116. As such, in this example, the liquid outlet 132 is configured to couple to the liquid disposal system 116 via the drain housing 250. However, in other examples, the separator housing 120 and/or the liquid outlet 132 can be additionally or alternatively coupled to the liquid disposal system 116 via a flexible drain tube.

In this arrangement, the suction device 110 can evacuate the surgical smoke and the liquid along the suction tube 118 to the smoke-liquid separator 112. At the smoke-liquid separator 112, the surgical smoke and the liquid can be received into the internal chamber 122 of the separator housing 120 through the inlet 124. The first suction source 128 of the smoke mitigation system 114 can apply the first suction force at the gas outlet 126 and evacuate the surgical smoke through the gas outlet 126 toward the smoke mitigation system 114. Locating the inlet 124 and the gas outlet 126 at an upper portion of the internal chamber 122 (e.g., at the top wall 220A) can help to efficiently evacuate the surgical smoke out of the internal chamber 122. At the smoke mitigation system 114, the surgical smoke can be filtered by the particulate filter 130 and/or vented to an external environment in which it does not pose a health risk.

The first suction force and/or the location of the gas outlet 126 can be configured such that the liquid flows downward (e.g., due to a force of gravity) and collects in the valve receptacle 242. While the amount of liquid in the internal chamber 122 and the valve receptacle 242 is less than the threshold amount, the spring 246 biases the first tube end 244A into contact with the wall 242A of the valve receptacle 242. Although the second suction source 134 of the liquid disposal system 116 is applying the second suction source to the liquid outlet 132, the second suction force is not applied to the internal chamber 122 due to the wall 242A blocking the first tube end 244A while the valve assembly 140 is in the closed state.

When the amount of the liquid in the internal chamber 122 and the valve receptacle 242 is greater than the threshold amount, the weight of the liquid overcomes the spring 246 and moves the valve receptacle 242 downward relative to the separator housing 120 and the tube 244. This results in a gap between the first tube end 244A and the wall 242A of the valve receptacle 242 (and automatically actuates the valve assembly 140 from the closed state to the open state). The second suction force is applied to the internal chamber 122 and evacuates the liquid through the tube 244 and out through the liquid outlet 132 toward the liquid disposal system 116. At the liquid disposal system 116, the liquid can be contained in the waste receptacle 136. After at least a portion of the liquid has been evacuated from the internal chamber 122 through the liquid outlet 132, the spring 246 can force the valve receptacle 242 upward relative to the separator housing 120 to automatically move the valve receptacle 242 back into contract with the first tube end 244A and thereby automatically actuate the valve assembly 140 back from the open state to the closed state.

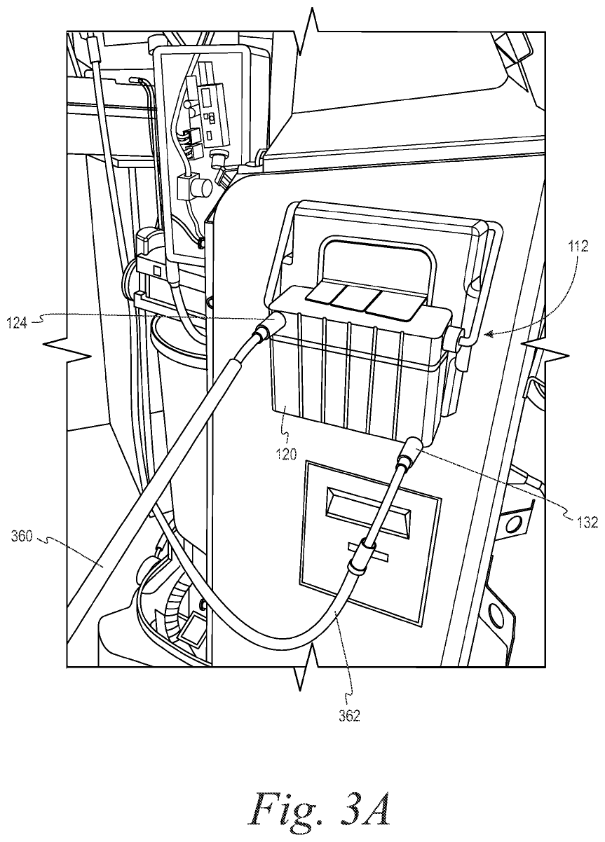

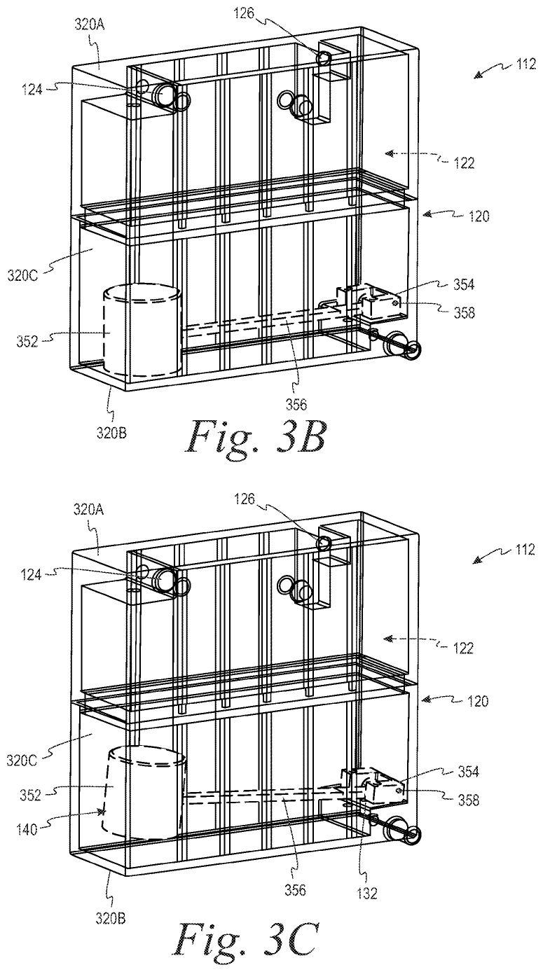

Referring now to FIGS. 3A-3C, an implementation of the smoke-liquid separator 112 is shown according to another example. In particular, FIG. 3A shows a perspective view of the smoke-liquid separator 112, FIG. 3B shows the smoke-liquid separator 112 of FIG. 3A with the valve assembly 140 in the closed state, and FIG. 3C shows the smoke-liquid separator 112 of FIG. 3A with the valve assembly 140 in the open state, according to the example.

As shown in FIGS. 3A-3C, the smoke-liquid separator 112 includes the separator housing 120 that defines the internal chamber 122. The separator housing 120 includes an inlet 124 that is configured to fluidly couple the internal chamber 122 to the suction device 110. The inlet 124 is configured to receive into the internal chamber 122 the liquid and the gas from the suction device 110. The separator housing 120 also includes the gas outlet 126 that is configured to output the gas from the internal chamber 122 to the smoke mitigation system 114, and the liquid outlet 132 that is configured to output the liquid from the internal chamber 122 to the liquid disposal system 116.

As shown in FIGS. 3A-3C, the separator housing 120 includes a top wall 320A, a bottom wall 320B, and a side wall 320C extending between the top wall 320A and the bottom wall 320B. As described above, the inlet 124 can be in an upper portion of the internal chamber 122, the gas outlet 126 can be in the upper portion of the internal chamber 122, and the liquid outlet 132 can be in a lower portion of the internal chamber 122. For instance, in FIGS. 3A-3C, the inlet 124 and the gas outlet 126 are in the side wall 320C at the upper portion of the internal chamber 122 above the liquid outlet 132 (e.g., adjacent to the top wall 320A) and the liquid outlet 132 is in the side wall 320C at the lower portion of the internal chamber 122 below the inlet 124 and the gas outlet 126 (e.g., adjacent to the bottom wall 320B). However, in other examples, the inlet 124 and/or the gas outlet 126 can be at respective apertures in the top wall 320A, and/or liquid outlet 132 can be an aperture in the bottom wall 320. With the inlet 124 located above the liquid outlet 132, the liquid moves from the inlet 124 toward the liquid outlet 132 due to a force of gravity.

As shown in FIGS. 3B-3C, the smoke-liquid separator 112 also includes the valve assembly 140 that is actuatable between (i) a closed state in which the valve assembly 140 inhibits the liquid from passing through the liquid outlet 132 (as shown in FIG. 3B), and (ii) an open state in which the valve assembly 140 allows the liquid to pass through the liquid outlet 132 (as shown in FIG. 3C). As described above, the valve assembly 140 is configured to: (i) automatically actuate to the closed state when an amount of the liquid in the internal chamber 122 is less than a threshold amount, and (ii) automatically actuate to the open state when the amount of the liquid in the internal chamber 122 is greater than the threshold amount.

In this example, the valve assembly 140 can include a float 352 in the internal chamber 122 that is configured to move between a first float position relative to the separator housing 120 (as shown in FIG. 3B) and a second float position relative to the separator housing 120 (as shown in FIG. 3C). As shown in FIGS. 3B-3C, the first position is lower than the second position (e.g., the float 352 is closer to the bottom wall 320B when the float 352 is in the first position as compared to when the float 352 is in the second position). The valve assembly 140 is in the open state when the float 352 is in the first float position shown in FIG. 3B, and the valve assembly 140 is in the closed state when the float 352 is in the second float position shown in FIG. 3C.

For example, in FIGS. 3B-3C, the valve assembly 140 can further include a plug 354 coupled to the float 352. The float 352 can be configured to move the plug 354 relative to the separator housing 120 between (i) a first plug position at which the liquid outlet 132 is covered by the plug 354 as shown in FIG. 3B, and (ii) a second plug position at which the liquid outlet 132 is at least partially uncovered as shown in FIG. 3C. Accordingly, the valve assembly 140 is in the open state when the float 352 is in the first float position and the plug 354 is in the first plug position, and the valve assembly 140 is in the closed state when the float 352 is in the second float position and the plug 354 is in the second plug position.

As shown in FIGS. 3B-3C, the valve assembly 140 can also include a float arm 356 that couples the float 352 and the plug 354. The plug 354 can be rotatably coupled to the separator housing 120 at a fulcrum 358. The first plug position of the plug 354 can include the plug 354 covering and/or blocking the liquid outlet 132, and the second plug position of the plug 354 can include the plug 354 rotated away from the liquid outlet 132 such that at least a portion of the liquid outlet 132 is exposed and not covered by the plug 354. The float arm 346 can help to increase leverage and make it easier to rotate the plug 354 as compared to other examples in which the plug 354 is coupled to the float 352 without the float arm 356.

In this arrangement, the suction device 110 can evacuate the surgical smoke and the liquid along the suction tube 118 to the smoke-liquid separator 112. At the smoke-liquid separator 112, the surgical smoke and the liquid can be received into the internal chamber 122 of the separator housing 120 through the inlet 124. The first suction source 128 of the smoke mitigation system 114 can apply the first suction force at the gas outlet 126 and evacuate the surgical smoke through the gas outlet 126 toward the smoke mitigation system 114. Locating the inlet 124 and the gas outlet 126 at an upper portion of the internal chamber 122 (e.g., at or near the top wall 320A) can help to efficiently evacuate the surgical smoke out of the internal chamber 122. At the smoke mitigation system 114, the surgical smoke can be filtered by the particulate filter 130 and/or vented to an external environment in which it does not pose a health risk.

The first suction force and/or the location of the gas outlet 126 can be configured such that the liquid flows downward (e.g., due to a force of gravity) and collects in the lower portion of the internal chamber 122. While the amount of liquid in the internal chamber 122 and the valve receptacle 242 is less than the threshold amount, the float 352 remains at the first float position and the plug 354 covers the liquid outlet 132. Although the second suction source 134 of the liquid disposal system 116 can be applying the second suction source to the liquid outlet 132, the second suction force is not applied to the internal chamber 122 due to the plug 354 blocking the liquid outlet 132 with the valve assembly 140 is in the closed state.

As the internal chamber 122 fills with liquid, the float 352 rises with the liquid due to a buoyancy of the float 352. When the amount of the liquid in the internal chamber 122 is greater than the threshold amount, the float 352 will have risen to the second float position shown in FIG. 3C. Responsive to the float 352 moving up from the first float position to the second float position, the float 352 moves the float arm 356, which rotates the plug 354 about the fulcrum 358 and away from the liquid outlet 132. This results in a gap between the plug 354 and the liquid outlet 132 when the valve assembly 140 is in the open state. The second suction force is applied to the internal chamber 122 and evacuates the liquid through the liquid outlet 132 toward the liquid disposal system 116. At the liquid disposal system 116, the liquid can be contained in the waste receptacle 136. After at least a portion of the liquid has been evacuated from the internal chamber 122 through the liquid outlet 132, the float 352 can move downward with the liquid, which rotates the plug 354 about the fulcrum 358 back towards the first plug position at which the plug 354 covers the liquid outlet 132. In this way, the valve assembly 140 can use the amount or level of the liquid in the internal chamber 122 to automatically actuate the valve assembly 140 between the closed state and the open state.

As shown in FIG. 3A, the smoke-liquid separator 112 can be configured to couple to the smoke mitigation system 114 in some examples. For instance, in FIGS. 3A-3C, an input connector tube 360 couples the inlet 124 to the suction device 110 (shown in FIG. 1), a drain tube 362 couples the liquid outlet 132 to the liquid disposal system 116 (shown in FIG. 1), and the gas outlet 126 is directly coupled to the particulate filter 130 (shown in FIG. 1) of the smoke mitigation system 114, according to one example. However, the smoke-liquid separator 112 can be standalone device, and/or the smoke-liquid separator 112 can be differently coupled to the suction device 110, the smoke mitigation system 114, and/or the liquid disposal system 116 in other examples.

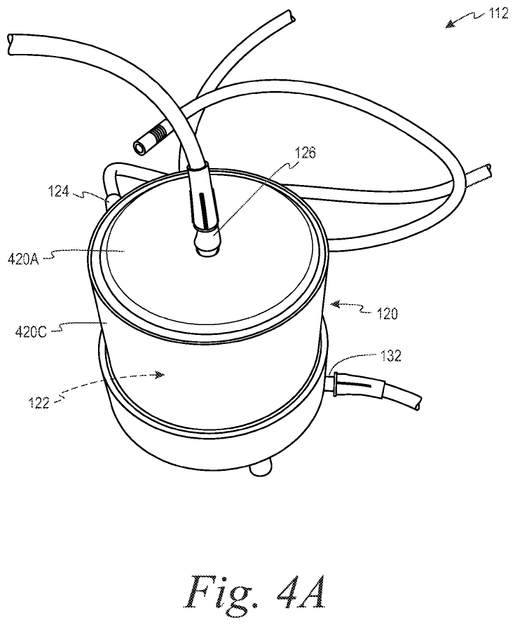

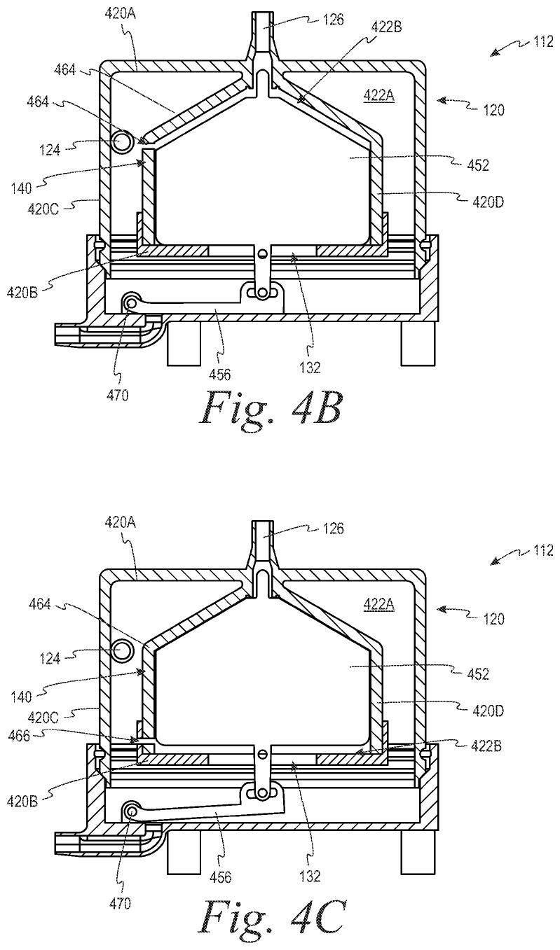

Referring now to FIGS. 4A-4C, an implementation of the smoke-liquid separator 112 is shown according to another example. In particular, FIG. 4A shows a perspective view of the smoke-liquid separator 112, FIG. 4B shows a cross-sectional view of the smoke-liquid separator 112 of FIG. 4A with the valve assembly 140 in the closed state, and FIG. 4C shows a cross-sectional view of the smoke-liquid separator 112 of FIG. 4A with the valve assembly 140 in the open state, according to the example.

As shown in FIGS. 4A-4C, the smoke-liquid separator 112 includes the separator housing 120 that defines the internal chamber 122. The separator housing 120 includes an inlet 124 that is configured to fluidly couple the internal chamber 122 to the suction device 110. The inlet 124 is configured to receive into the internal chamber 122 the liquid and the gas from the suction device 110. The separator housing 120 also includes the gas outlet 126 that is configured to output the gas from the internal chamber 122 to the smoke mitigation system 114, and the liquid outlet 132 that is configured to output the liquid from the internal chamber 122 to the liquid disposal system 116.

As shown in FIGS. 4A-4C, the separator housing 120 includes a top wall 420A, a bottom wall 420B, and a side wall 420C extending between the top wall 420A and the bottom wall 420B. As described above, the inlet 124 can be in an upper portion of the internal chamber 122, the gas outlet 126 can be in the upper portion of the internal chamber 122, and the liquid outlet 132 can be in a lower portion of the internal chamber 122. For instance, in FIGS. 4A-4C, the inlet 124 is in the side wall 420C at the upper portion of the internal chamber 122 above the liquid outlet 132, the gas outlet 126 is in the top wall 420A, and the liquid outlet 132 is in the bottom wall 420B (e.g., below the inlet 124 and the gas outlet 126). However, in other examples, the inlet 124, the gas outlet 126, and/or the liquid outlet 132 can be at respective apertures in different portions of the separator housing 120. With the inlet 124 located above the liquid outlet 132, the liquid moves from the inlet 124 toward the liquid outlet 132 due to a force of gravity.

As shown in FIGS. 4B-4C, the smoke-liquid separator 112 also includes the valve assembly 140 that is actuatable between (i) a closed state in which the valve assembly 140 inhibits the liquid from passing through the liquid outlet 132 (as shown in FIG. 4B), and (ii) an open state in which the valve assembly 140 allows the liquid to pass through the liquid outlet 132 (as shown in FIG. 4C). As described above, the valve assembly 140 is configured to: (i) automatically actuate to the closed state when an amount of the liquid in the internal chamber 122 is less than a threshold amount, and (ii) automatically actuate to the open state when the amount of the liquid in the internal chamber 122 is greater than the threshold amount.

In this example, the valve assembly 140 can include a float 452 in the internal chamber 122 that is configured to move between a first float position relative to the separator housing 120 (as shown in FIG. 4B) and a second float position relative to the separator housing 120 (as shown in FIG. 4C). As shown in FIGS. 4B-4C, the first position is lower than the second position (e.g., the float 452 is closer to the bottom wall 420B when the float 452 is in the first position as compared to when the float 452 is in the second position). The valve assembly 140 is in the open state when the float 452 is in the first float position shown in FIG. 4B, and the valve assembly 140 is in the closed state when the float 452 is in the second float position shown in FIG. 4C.

As shown in FIG. 4B, the float 452 blocks the liquid outlet 132 when the float 452 is in the first float position. For example, the float 452 can have a size that is greater than a size of the liquid outlet 132, and the float 452 can cover the liquid outlet 132. In some examples, the float 452 can contact and rest on the bottom wall 420B of the separator housing 120.

As shown in FIG. 4C, the float 452 unblocks the liquid outlet 132 when the float 452 is in the second float position. For instance, when the liquid is received in the internal chamber 122 from the inlet 124, the liquid can fill the internal chamber 122 can cause the float 452 to rise with the liquid in an upward direction (e.g., a direction from the bottom wall 420B toward the top wall 420A) and away from the liquid outlet 132 at the bottom wall 420B. As a result, when the valve assembly 140 is in the open state, a gap is formed between the float 452 and the liquid outlet 132 (and/or the bottom wall 420B). This gap allows the liquid to pass from the internal chamber 122 through the liquid outlet 132. Additionally, the gap between the float 452 and the liquid outlet 132 allows the second suction source 134 to apply the second suction force to the internal chamber 122 through the liquid outlet 132 to assist in evacuating the liquid from the internal chamber 122.

In the example shown in FIGS. 4B-4C, the separator housing 120 can include one or more inner walls 420D that divide the internal chamber 122 into a first sub-chamber 422A and a second sub-chamber 422B. The float 452 can be positioned and movable in the second sub-chamber 422B. The inlet 124 can be in fluid communication with the first sub-chamber 422A. The gas outlet 126 in fluid communication with the second sub-chamber 422B. The one or more inner walls 420D can include a first aperture 464 and a second aperture 466 that is configured to fluidly couple the first sub-chamber 422A and the second sub-chamber 422B. The second aperture 466 can be lower than the first aperture 464 to allow the liquid to pass from the first sub-chamber 422A to interact with the float 452 in the second sub-chamber 422B (e.g., to raise and lower the float 452 in accordance with the amount of liquid in the internal chamber 122).

When the float 452 is in the first float position shown in FIG. 4B, the float 452 (i) unblocks the first aperture 464 to permit the surgical smoke to flow from the first sub-chamber 422A to the second sub-chamber 422B and out of the gas outlet 126, and (ii) blocks the liquid outlet 132. When the float 452 is in the second float position as shown in FIG. 4C, the float 452 (i) blocks the first aperture 464 to prevent the surgical smoke from flowing from the first sub-chamber 422A to the second sub-chamber 422B, and (ii) unblocks the liquid outlet 132. Accordingly, in this example, the valve assembly 140 can be configured to selectively evacuate only one of the surgical smoke or evacuate the liquid at a time.

As shown in FIGS. 4A-4B, the separator housing 120 can be coupled to a drain housing 468. In this example, the liquid outlet 132 fluidly couples the internal chamber 122 of the separator housing 120 and an internal cavity of the drain housing 468 when the float 452 is in the second float position (and the valve assembly 140 is in the open state). For instance, the drain housing 468 can be below the separator housing 120. The drain housing 468 can help to facilitate a coupling between the liquid outlet 132 and the liquid disposal system 116.

In some examples that include the drain housing 468, as shown in FIGS. 4B-4C, the valve assembly 140 can also include a float arm 456 that couples the float 452 to an anchor point 470 on the drain housing 468. The float arm 456 can help to create leverage, which can help to reduce a force required to open the valve assembly 468 due to the buoyancy of the float 452. In some examples, the float arm 456 can additionally or alternatively cover an outlet from the drain housing 468 when the float 452 is in the first float position, and uncover the outlet from the drain housing 468 when the float 452 is in the second float position.

In this arrangement, the suction device 110 can evacuate the surgical smoke and the liquid along the suction tube 118 to the smoke-liquid separator 112. At the smoke-liquid separator 112, the surgical smoke and the liquid can be received into the internal chamber 122 of the separator housing 120 through the inlet 124. The first suction source 128 of the smoke mitigation system 114 can apply the first suction force at the gas outlet 126 and evacuate the surgical smoke through the first aperture 464 and out the gas outlet 126 toward the smoke mitigation system 114. Locating the inlet 124 and the gas outlet 126 at an upper portion of the internal chamber 122 (e.g., at or near the top wall 320A) can help to efficiently evacuate the surgical smoke out of the internal chamber 122. At the smoke mitigation system 114, the surgical smoke can be filtered by the particulate filter 130 and/or vented to an external environment in which it does not pose a health risk.

The first suction force and/or the location of the gas outlet 126 can be configured such that the liquid flows downward (e.g., due to a force of gravity) and collects in the lower portion of the internal chamber 122. While the amount of liquid in the internal chamber 122 and the valve receptacle 242 is less than the threshold amount, the float 452 remains at the first float position and the float 452 covers the liquid outlet 132. Although the second suction source 134 of the liquid disposal system 116 can be applying the second suction source to the liquid outlet 132, the second suction force is not applied to the internal chamber 122 due to the float 452 blocking the liquid outlet 132 with the valve assembly 140 is in the closed state.

As the internal chamber 122 fills with liquid, the liquid passes through the second aperture 466 and causes the float 452 to rise due to a buoyancy of the float 352. When the amount of the liquid in the internal chamber 122 is greater than the threshold amount, the float 452 will have risen to the second float position shown in FIG. 4C. Responsive to the float 352 moving up from the first float position to the second float position, the float 452 blocks the first aperture 464 inhibiting or preventing further evacuation of the surgical smoke and/or application of the first suction force to the internal chamber 122. Additionally, when the float 452 is in the second float position, there is a gap between the float 452 and the liquid outlet 132 when the valve assembly 140 is in the open state. The second suction force is applied to the internal chamber 122 and evacuates the liquid through the liquid outlet 132 toward the liquid disposal system 116. At the liquid disposal system 116, the liquid can be contained in the waste receptacle 136. After at least a portion of the liquid has been evacuated from the internal chamber 122 through the liquid outlet 132, the float 452 can move downward with the liquid, which causes the float 452 to unblock the first aperture 464 and block the liquid outlet 132. In this way, the valve assembly 140 can use the amount or level of the liquid in the internal chamber 122 to automatically actuate the valve assembly 140 between the closed state and the open state.

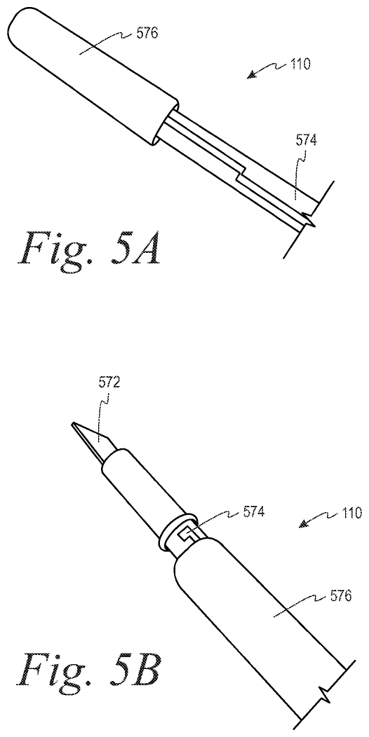

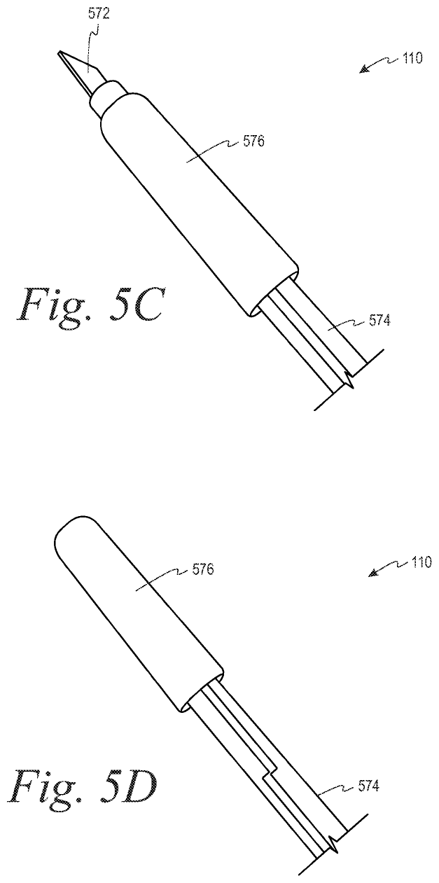

Referring now to FIG. 5A-5D, implementations of the suction device 110 is shown according to some examples. FIG. 5A depicts a first implementation of the suction device 110 with a suction sleeve in an extended position, FIG. 5B depicts the suction device 110 of FIG. 5A with the suction sleeve in a retracted position, FIG. 5C depicts a second implementation of the suction device 110 with an electrosurgical electrode in an extended position, and FIG. 5D depicts the suction device 110 of FIG. 5C with the electrosurgical electrode in an retracted position, according to the examples.

As described above, in some examples, the suction device 110 can be a medical device that is configured to create the surgical smoke during a surgical procedure. As examples, FIGS. 5A-5D depict an suction device 110 that can (i) evacuate the surgical smoke and the liquid through the suction tube 118, and (ii) apply a radiofrequency (RF) energy to tissue at the surgical site to cut and/or coagulate tissue.

In FIGS. 5A-5D, the suction device 110 includes an electrosurgical electrode 572 that can radiofrequency (RF) energy to tissue at the surgical site to cut and/or coagulate tissue. The electrosurgical electrode 572 extends from a distal end of a shaft 574. The suction device 110 can also include a plurality of electrical components that facilitate supplying the RF energy, which the suction device 110 receives from an electrosurgical generator, to the electrosurgical electrode 572. For example, the suction device 110 can include at least one electrical component selected from a group of electrical components including: a printed circuit board (e.g., a flexible printed circuit board), one or more conductors that are configured to conduct RF energy from a power cord to the electrosurgical electrode, and/or one or more control switches. One or more of the electrical components can be positioned in a housing (e.g., a handle or the shaft 574). To mitigate (or prevent) damage to the electrical components of the suction device 110, the suction tube 118 can be sealed (e.g., by one or more rubber seals and/or potting) to mitigate (or prevent) the surgical smoke and/or the liquid from contacting the electrical components.

Additionally, to help evacuate the liquid, the suction device 110 can include a suction sleeve 576 that can move relative to the shaft 574 and the electrosurgical electrode 572. For example, in FIGS. 5A-5B, the suction sleeve 576 can move between (i) an extended position relative to the shaft 574 (shown in FIG. 5A) at which a distalmost end of the suction sleeve 576 is located distally of a distalmost end of the electrosurgical electrode 572 and (ii) a retracted position relative to the shaft 574 (shown in FIG. 5B) at which at least a portion of the electrosurgical electrode 572 extends distally of the distalmost end of the suction sleeve 576. When the suction sleeve 576 is in the extended position, the suction sleeve 576 can be submerged in the liquid to better evacuate the liquid from the surgical site towards the smoke-liquid separator 112. When the suction sleeve 576 is in the retracted position, the electrosurgical electrode 572 can be exposed to perform an electrosurgical operation.

In the example of FIGS. 5C-5D, the suction sleeve 576 can remain fixed relative to the shaft 574 and the electrosurgical electrode 572 can move relative to the shaft 574 and the suction sleeve 576. For example, in FIGS. 5C-5D, the electrosurgical electrode 572 can move between (i) an extended position relative to the shaft 574 (shown in FIG. 5C) at which at least a portion of the electrosurgical electrode 572 is located distally of a distalmost end of the suction sleeve 576 and (ii) a retracted position relative to the shaft 574 (shown in FIG. 5D) at which an entirety of the electrosurgical electrode 572 is proximal of a distalmost end of the suction sleeve 576. When the electrosurgical electrode 572 is in the extended position, the electrosurgical electrode 572 can be exposed to perform an electrosurgical operation. When the electrosurgical electrode 572 is in the retracted position, the suction sleeve 576 can be submerged in the liquid to better evacuate the liquid from the surgical site towards the smoke-liquid separator 112.

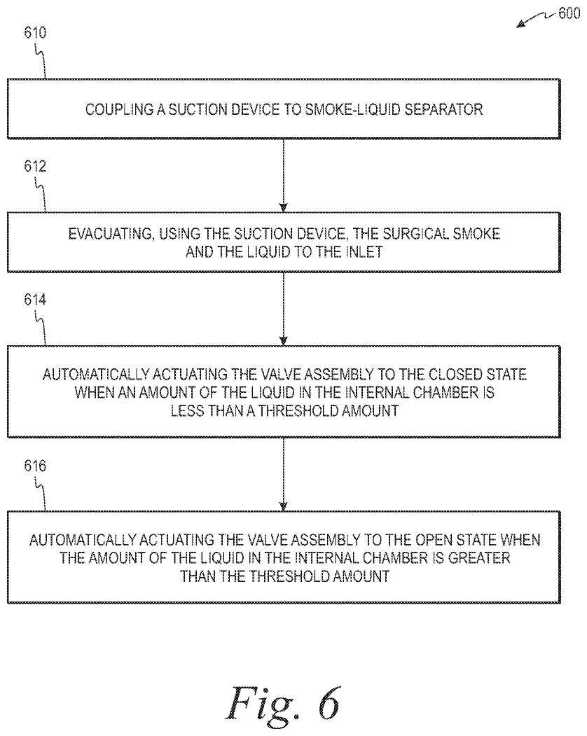

Referring now to FIG. 6, a flowchart for a process 600 of evacuating a surgical smoke and a liquid is shown, according to an example. As shown at block 610, the process 600 can include coupling a suction device to smoke-liquid separator. The smoke-liquid separator can include a separator housing that defines and internal chamber. The separator housing can include an inlet configured to fluidly couple the internal chamber to a suction device. The inlet is configured to receive into the internal chamber a liquid and a gas from the suction device. The separator housing can also include a gas outlet configured to output the gas from the internal chamber to a smoke mitigation system, and a liquid outlet configured to output the liquid from the internal chamber to a liquid disposal system. The smoke-liquid separator can further include a valve assembly that is actuatable between (i) a closed state in which the valve assembly inhibits the liquid from passing through the liquid outlet, and (ii) an open state in which the valve assembly allows the liquid to pass through the liquid outlet.

At block 612, the process 600 can include evacuating, using the suction device, the surgical smoke and the liquid to the inlet. At block 614, the process 600 can include automatically actuating the valve assembly to the closed state when an amount of the liquid in the internal chamber is less than a threshold amount. At block 616, the process 600 can includes automatically actuating the valve assembly to the open state when the amount of the liquid in the internal chamber is greater than the threshold amount.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may provide different advantages as compared to other advantageous embodiments. The implementation or implementations selected are chosen and described in order to best explain the principles of the embodiments, the practical application, and to enable others of ordinary skill in the art to understand the disclosure for various embodiments with various modifications as are suited to the particular use contemplated.

What is claimed is:

1. A smoke and liquid separator device, comprising:
a separator housing that defines an internal chamber, wherein the separator housing comprises:
an inlet configured to fluidly couple the internal chamber to a suction device, wherein the inlet is configured to receive into the internal chamber a liquid and a gas from the suction device,
a gas outlet configured to output the gas from the internal chamber to a smoke mitigation system, and
a liquid outlet configured to output the liquid from the internal chamber to a liquid disposal system; and
a valve assembly that is actuatable between (i) a closed state in which the valve assembly inhibits the liquid from passing through the liquid outlet, and (ii) an open state in which the valve assembly allows the liquid to pass through the liquid outlet,
wherein the valve assembly is configured to: (i) automatically actuate to the closed state when an amount of the liquid in the internal chamber is less than a threshold amount, and (ii) automatically actuate to the open state when the amount of the liquid in the internal chamber is greater than the threshold amount.

2. The smoke and liquid separator device of claim 1, wherein the valve assembly further comprises a valve receptacle that is disposed in the internal chamber,
wherein the valve receptacle is movable relative to the separator housing,
wherein, in a first position of the valve receptacle relative to the separator housing, the valve assembly is in the closed state, and
wherein, in a second position of the valve receptacle relative to the separator housing, the valve assembly is in the open state.

3. The smoke and liquid separator device of claim 2, wherein the valve assembly further comprises a spring that biases the valve receptacle towards the first position relative to the separator housing.

4. The smoke and liquid separator device of claim 3, wherein, when the amount of the liquid in the internal chamber is greater than the threshold amount, a weight of the liquid and the valve receptacle overcomes a biasing force applied by the spring between the separator housing and the valve receptacle.

5. The smoke and liquid separator of any one of claims 2-4, wherein the valve assembly further comprises a tube that extends between a first tube end and a second tube end,
wherein the first tube end is in the internal chamber of the separator housing and the second tube end is in communication with the liquid outlet,
wherein, when the valve assembly is in the closed state, a wall of the valve receptacle contacts the first tube end, and
wherein, when the valve assembly is in the open state, the first tube end is spaced apart from the wall of the valve receptacle.

6. The smoke and liquid separator of claim 5, wherein the tube is fixedly coupled to the separator housing such that the valve receptacle moves relative to the tube when the valve receptacle moves relative to the separator housing.

7. The smoke and liquid separator of any one of claims 5-6, wherein the valve assembly further comprises one or more O-ring seals at an interface between the first tube end and the wall of the valve receptacle when the valve receptacle is in the first position relative to the separator housing.

8. The smoke and liquid separator of any one of claims 5-7, wherein the tube comprises a bend between the first tube end and the second tube end.

9. The smoke and liquid separator of any one of claims 5-8, wherein the separator housing comprises a top wall, a bottom wall, and a side wall extending between the top wall and the bottom wall, wherein the inlet and the gas outlet are at the top wall, and wherein the liquid outlet is at the bottom wall, and wherein the liquid moves from the inlet toward the liquid outlet due to a force of gravity.

10. The smoke and liquid separator of claim 9, wherein the tube extends through the bottom wall.

11. The smoke and liquid separator of claim 1, wherein the valve assembly comprises a float in the internal chamber that is configured to move between a first float position and a second float position relative to the separator housing,
wherein the valve assembly is in the open state when the float is in the first float position, and
wherein the valve assembly is in the closed state when the float is in the second float position.

12. The smoke and liquid separator of claim 11, wherein the valve assembly further comprises a plug,
wherein the float is configured to move the plug relative to the separator housing between (i) a first plug position at which the liquid outlet is covered by the plug, and (ii) a second plug position at which the liquid outlet is uncovered,
wherein the valve assembly is in the open state when the float is in the first float position and the plug is in the first plug position, and
wherein the valve assembly is in the closed state when the float is in the second float position and the plug is in the second plug position.

13. The smoke and liquid separator of claim 12, wherein the valve assembly further comprises a float arm that couples the float and the plug.

14. The smoke and liquid separator of claim 13, wherein the plug is rotatably coupled to the separator housing at a fulcrum.

15. The smoke and liquid separator of any one of claims 12-14, wherein the inlet is in an upper portion of the internal chamber, the gas outlet is in an upper portion of the internal chamber, and the liquid outlet is in a lower portion of the internal chamber.

16. The smoke and liquid separator of claim 11, wherein the float blocks the liquid outlet when the float is in the first float position, and the float unblocks the liquid outlet when the float is in the second float position.

17. The smoke and liquid separator of claim 16, wherein the separator housing comprises one or more inner walls that divide the internal chamber into a first sub-chamber and a second sub-chamber,
wherein the float is positioned and movable in the second sub-chamber,
wherein the inlet is in fluid communication with the first sub-chamber,
wherein the gas outlet is in fluid communication with the second sub-chamber,
wherein the one or more inner walls comprise a first aperture that is configured to fluidly couple the first sub-chamber and the second sub-chamber,
wherein, when the float is in the first float position, the float unblocks the first aperture to permit the gas to flow from the first sub-chamber to the second sub-chamber and out of the gas outlet, and
wherein, when the float is in the second float position, the float blocks the first aperture to prevent the gas from flowing from the first sub-chamber to the second sub-chamber.

18. The smoke and liquid separator of claim 17, wherein the one or more inner walls comprise a second aperture that is configured to fluidly couple the first sub-chamber and the second sub-chamber, wherein the second aperture is lower than the first aperture to allow the liquid to pass from the first sub-chamber to interact with the float in the second sub-chamber.

19. The smoke and liquid separator of any one of claims 16-18, wherein the separator housing is coupled to a drain housing, and
wherein the liquid outlet fluidly couples the internal chamber of the separator housing and an internal cavity of the drain housing when the float is in the second float position.

20. The smoke and liquid separator of claim 19, wherein the valve assembly comprises a float arm that couples the float to an anchor point on the drain housing.

21. A method of evacuating a surgical smoke and a liquid, comprising:
coupling a suction device to smoke-liquid separator, wherein the smoke-liquid separator comprises:
(i) a separator housing that defines an internal chamber, wherein the separator housing comprises:
(a) an inlet configured to fluidly couple the internal chamber to a suction device, wherein the inlet is configured to receive into the internal chamber a liquid and a gas from the suction device,
(b) a gas outlet configured to output the gas from the internal chamber to a smoke mitigation system, and
(c) a liquid outlet configured to output the liquid from the internal chamber to a liquid disposal system; and
(ii) a valve assembly that is actuatable between (i) a closed state in which the valve assembly inhibits the liquid from passing through the liquid outlet, and (ii) an open state in which the valve assembly allows the liquid to pass through the liquid outlet,
evacuating, using the suction device, the surgical smoke and the liquid to the inlet;
automatically actuating the valve assembly to the closed state when an amount of the liquid in the internal chamber is less than a threshold amount; and
automatically actuating the valve assembly to the open state when the amount of the liquid in the internal chamber is greater than the threshold amount.
